(19) United States
(12) Patent Application Publication
(10) Pub. No.: US 2024/0423452 A1
(43) Pub. Date: Dec. 26, 2024

(54) ENDOSCOPE INSERTION ASSISTING SYSTEM, ENDOSCOPE INSERTION ASSISTING METHOD, AND STORAGE MEDIUM

(71) Applicant: OLYMPUS MEDICAL SYSTEMS CORP., Tokyo (JP)

(72) Inventors: Naoki FUKATSU, Tokyo (JP); Hiromasa FUJITA, Tokyo (JP); Kensuke MIYAKE, Tokyo (JP)

(73) Assignee: OLYMPUS MEDICAL SYSTEMS CORP., Tokyo (JP)

(21) Appl. No.: 18/828,486

(22) Filed: Sep. 9, 2024

Related U.S. Application Data
(63) Continuation of application No. PCT/JP2022/012902, filed on Mar. 18, 2022.

(51) Int. Cl. A61B 1/00; A61B 1/005

(52) U.S. Cl. CPC A61B 1/00096; A61B 1/0055

(57) ABSTRACT

An endoscopic image acquisition unit acquires shape information of an endoscope insertion part during endoscopic examination. According to a priority condition, executed is at least either: a process by an insertion status estimation unit entering the shape information to an insertion status learning model, thereby acquiring, from the insertion status learning model, an insertion status category resulted from categorization of insertion status of the endoscope insertion part; or, a process by a shape determination unit applying the shape information to a shape category determination logic, thereby acquiring an insertion shape category resulted from categorization of insertion shape of the endoscope insertion part.

### CROSS-REFERENCE TO RELATED APPLICATIONS

This application is based upon and claims the benefit of priority from the International Application No. PCT/JP2022/012902, filed on Mar. 18, 2022, the entire contents of which are incorporated.

### BACKGROUND

### 1. Technical Field

The present disclosure relates to an endoscope insertion assisting system, an endoscope insertion assisting method, and a storage medium, for assisting insertion of an endoscope.

### 2. Description of the Related Art

For use in colonoscopy, there is known an endoscope control device that uses AI to classify shape of an insertion part of a colonoscope, and to control insertion of the colonoscope with reference to a result of classification.

### SUMMARY

An endoscope insertion assisting system according to an aspect of the present disclosure includes one or more processors having hardware. The processor is structured to acquire shape information of an endoscope insertion part during endoscopic examination; and to execute, according to a priority condition, at least either one of entering the shape information to an insertion status learning model, thereby acquiring, from the insertion status learning model, an insertion status category resulted from categorization of insertion status of the endoscope insertion part; and applying the shape information to a shape category determination logic, thereby acquiring an insertion shape category resulted from categorization of insertion shape of the endoscope insertion part.

Another aspect of the present disclosure relates to a method for assisting endoscope insertion. The method includes acquiring shape information of an endoscope insertion part during endoscopic examination; and executing, according to a priority condition, at least either one of entering the shape information to an insertion status learning model, thereby acquiring, from the insertion status learning model, an insertion status category resulted from categorization of insertion status of the endoscope insertion part; and applying the shape information to a shape category determination logic, thereby acquiring an insertion shape category resulted from categorization of insertion shape of the endoscope insertion part.

Note that also free combinations of these constituents, and also any of the expressions of the present disclosure exchanged among the method, apparatus, system, storage medium, computer program product and so forth, are valid as the embodiments of the present disclosure.

### BRIEF DESCRIPTION OF THE DRAWINGS

FIG. 1 is a diagram illustrating an overall system structure involved in colonoscopy according to an embodiment;

FIG. 2 is a diagram illustrating an exemplary endoscope used in this embodiment;

FIG. 3 is a diagram illustrating an exemplary structure of an endoscope insertion assisting system according to First Embodiment;

FIG. 4 is a drawing illustrating a scene of colonoscopy;

FIG. 5 is a diagram illustrating an exemplary screen that appears on a display device during endoscopic examination;

FIGS. 6A and 6B are drawings explaining an exemplary determination logic used in a shape determination unit;

FIGS. 7A and 7B are drawings explaining Correction Example 1 of an insertion status category;

FIGS. 8A and 8B are drawings explaining Correction Example 2 of the insertion status category;

FIG. 9 is a flow chart illustrating an exemplary operation of the endoscope insertion assisting system according to First Embodiment;

FIG. 10 is a drawing illustrating an exemplary structure of an endoscope insertion assisting system according to Second Embodiment;

FIGS. 11A and 11B are drawings illustrating exemplary endoscope shapes before and after posture change;

FIG. 12 is a flow chart illustrating an exemplary operation of an endoscope insertion assisting system according to Second Embodiment;

FIG. 13 is a drawing illustrating an exemplary structure of an endoscope insertion assisting system according to Third Embodiment;

FIG. 14 is a flow chart illustrating an exemplary operation of the endoscope insertion assisting system according to Third Embodiment;

FIG. 15 is a drawing illustrating an exemplary structure of an endoscope insertion assisting system according to Fourth Embodiment;

FIG. 16 is a flow chart illustrating an exemplary operation of the endoscope insertion assisting system according to Fourth Embodiment; and

FIG. 17 is a drawing illustrating an exemplary structure of an endoscope insertion assisting system according to Fifth Embodiment.

### DETAILED DESCRIPTION

The invention will now be described by reference to the preferred embodiments. This does not intend to limit the scope of the present invention, but to exemplify the invention.

This embodiment relates to colonoscopy. High-quality colonoscopy with less physical burden on the patient needs a highly sophisticated technique. Aiming at enabling high-quality colonoscopy, a system has been developed, in which AI recognizes how an insertion part of an endoscope deforms in the body, and determines an optimum operation method in such insertion status. If the AI unfortunately misrecognized the insertion status of the endoscope, the system would present an erroneous operation method to a doctor, or would send erroneous operation information to a control system of an automatic insertion device.

In order to reduce the risk, this embodiment avoids employment of one determination result of the AI simply as it is, but combines the result with a result of shape recognition by a logic, or with a determination result of other AI, thus introducing a scheme of reducing the misrecognition of the insertion status.

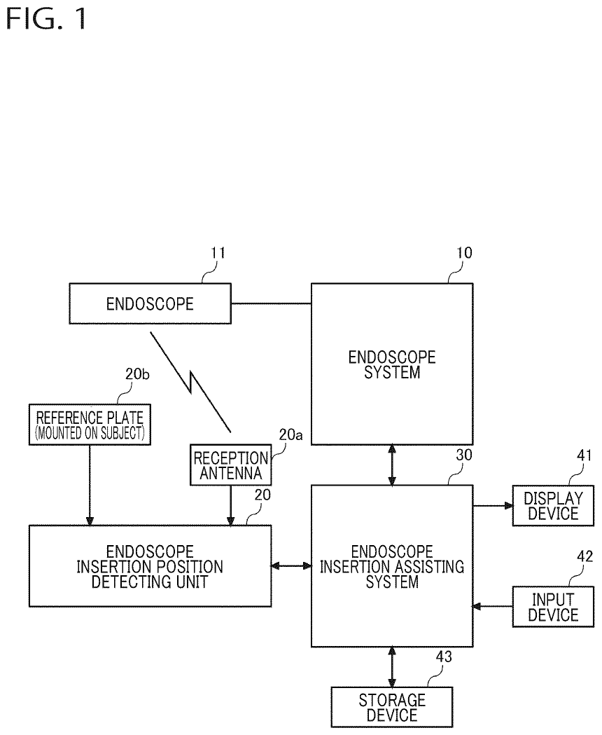

FIG. 1 is a diagram illustrating an overall system structure involved in colonoscopy according to an embodiment. This embodiment uses an endoscope system 10, an endoscope 11, a light source device 15, an endoscope insertion position detecting unit (UPD) 20, an endoscope insertion assisting system 30, a display device 41, an input device 42, and a storage device 43. The endoscope 11 in this embodiment is a colonoscope to be inserted into a large intestine of a subject (patient).

The endoscope 11 includes a lens and a solid-state imaging element (for example, CMOS image sensor, CCD image sensor, or CMD image sensor). The solid-state imaging element converts light condensed by the lens to an electric signal, and outputs an endoscopic image (electric signal) to the endoscope system 10. The endoscope 11 includes a forceps channel. With a treatment tool inserted through the forceps channel, an operator (doctor) can take part in various treatments during endoscopic examination.

The light source device 15 includes a light source such as a xenon lamp, and supplies observation light (white light, narrow band light, fluorescence, near infrared light, etc.) to the distal end of the endoscope 11. The light source device 15 also has, built therein, a pump that feeds water or air to the endoscope 11.

The endoscope system 10 controls the light source device 15, as well as processes an endoscopic image input from the endoscope 11. The endoscope system 10 has functions such as narrow band imaging (NBI), red dichromatic imaging (RDI), texture and color enhancement imaging (TXI), and extended depth of field (EDOF), for example.

From the narrow band imaging, which involves irradiation of lights of specific wavelengths at violet (415 nm) and green (540 nm) strongly absorbed by hemoglobin in blood, obtainable is an endoscopic image with emphasized capillaries and microstructures of supermucosal layer. From the red dichromatic imaging, which involves irradiation of lights of specific wavelengths in three colors (green, amber, and red), obtainable is an endoscopic image with emphasized contrast of deep tissue. From the structural color enhancement imaging, obtainable is an endoscopic image in which three elements of “structure”, “color tone”, and “brightness” of mucosal surface, observed under normal light, are optimized. From the extended depth of field, which involves synthesis of two images individually focused on near point and distant point, obtainable is an endoscopic image with a wide focal range.

The endoscope system 10 outputs an endoscopic image processed from the endoscopic image input from the endoscope 11, or the endoscopic image input from the endoscope 11 as it is, to the endoscope insertion assisting system 30.

The endoscope insertion position detecting unit (UPD) 20 is a device for observing a three-dimensional shape of the endoscope 11 inserted into a lumen of the subject. The endoscope insertion position detecting unit 20 has connected thereto a reception antenna 20a. The reception antenna 20a is an antenna for detecting a magnetic field generated by a plurality of magnetic coils built in the endoscope 11.

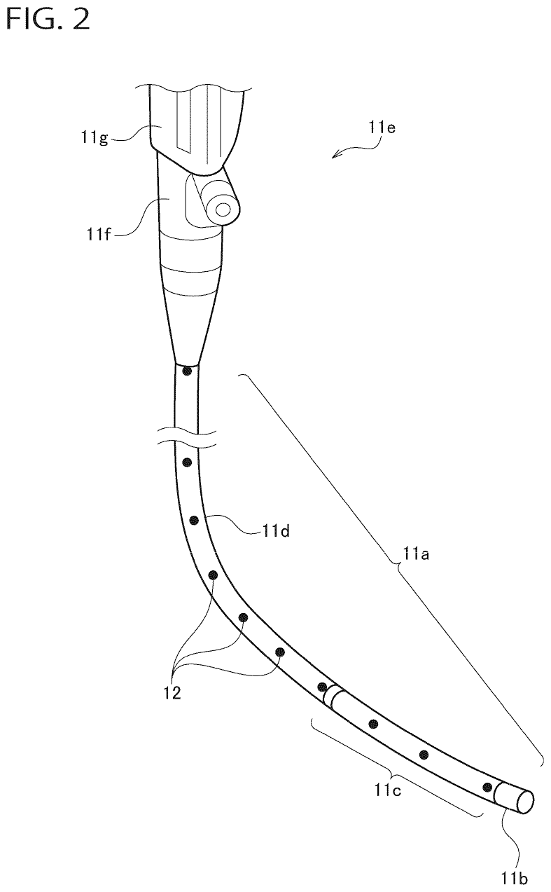

FIG. 2 is a diagram illustrating an example of the endoscope 11 used in this embodiment. The endoscope 11 has an elongated tubular insertion part 11a formed of a flexible member, and an operation unit 11e connected to the proximal end of the insertion part 11a. The insertion part 11a, when viewed from the distal end towards the proximal end, a distal end rigid part 11b, a bending section 11c, and a flexible tube 11d. The proximal end of the distal end rigid part 11b is connected to the distal end of the bending section 11c, and the proximal end of the bending section 11c is connected to the proximal end of the flexible tube 11d.

The operation unit 11e has a main body 11f from which the flexible tube 11d extends, and a grip 11g connected to the proximal end of the main body 11f. The grip 11g is gripped by the operator. The operation unit 11e has, extended therefrom, a universal cord that contains an imaging electric cable extended from inside of the insertion part 11a, a light guide and so forth, which is connected to the endoscope system 10 and the light source device 15.

The distal end rigid part 11b is a distal end part of the insertion part 11a, and is also a distal-end part of the endoscope 11. The distal end rigid part 11b has built therein a solid-state imaging element, an illumination optical system, an observation optical system and so forth. Light emitted from the light source device 15 propagates in the light guide to reach the distal end face of the distal end rigid part 11b, and is emitted from the distal end face of the distal end rigid part 11b to illuminate a target to be observed in the lumen.

The bending section 11c is formed of node rings connected in the longitudinal direction of the insertion part 11a. The bending section 11c bends in a desired direction in response to operator's operation entered through the operation unit 11e, wherein the bending causes changes in position and direction of the distal end rigid part 11b.

The flexible tube 11d is a tubular member that extends from the main body 11f of the operation unit 11e, having a desired flexibility, and is bendable under external force. The operator inserts the insertion part 11a into the large intestine of the subject, while bending the bending section 11c or twisting the flexible tube 11d.

The insertion part 11a has, inside thereof, a plurality of magnetic coils 12 arranged at predetermined intervals (10 cm intervals, for example) in the longitudinal direction. Each magnetic coil 12 generates a magnetic field upon being energized. The plurality of magnetic coils 12 functions as position sensors for detecting the individual positions of the insertion part 11a.

Referring now back to FIG. 1. The reception antenna 20a receives the magnetic fields emitted from the plurality of magnetic coils 12 built in the insertion part 11a of the endoscope 11, and outputs the result to the endoscope insertion position detecting unit 20. The endoscope insertion position detecting unit 20 applies the magnetic field intensity of the individual magnetic coils 12 received by the reception antenna 20a to a predetermined position detection algorithm, to estimate three-dimensional positions of the individual magnetic coils 12. The endoscope insertion position detecting unit 20 subjects the thus estimated three-dimensional positions of the individual magnetic coils 12 to curve interpolation, to generate a three-dimensional endoscope shape of the insertion part 11a of the endoscope 11.

A reference plate 20b is attached to a subject (the abdomen of the subject, for example). The reference plate 20b has arranged thereon a posture sensor for detecting the posture of the subject. To the posture sensor, applicable is a three-axis acceleration sensor or a gyro sensor for example. In FIG. 1, the reference plate 20b is connected by a cable to the endoscope insertion position detecting unit 20, and outputs three-dimensional posture information that represents the posture of the reference plate 20b (that is, the posture of the subject) to the endoscope insertion position detecting unit 20.

Note that the posture sensor arranged on the reference plate 20b may be a plurality of magnetic coils similar to the magnetic coils 12 built in the insertion part 11a of the endoscope 11. The reception antenna 20a in this case receives the magnetic fields emitted from the plurality of magnetic coils arranged on the reference plate 20b, and outputs the result to the endoscope insertion position detecting unit 20. The endoscope insertion position detecting unit 20 applies the magnetic field intensity of the individual magnetic coils received by the reception antenna 20a to a predetermined position detection algorithm, to generate three-dimensional information that represents the posture of the reference plate 20b (that is, the posture of the subject).

The endoscope insertion position detecting unit 20 changes the generated three-dimensional endoscope shape so as to follow the change in the three-dimensional posture information. More specifically, the endoscope insertion position detecting unit 20 changes the three-dimensional endoscope shape, so as to cancel the change in the three-dimensional posture information. This makes it possible to recognize the endoscope shape always from a specific viewpoint (such as a viewpoint from which the abdomen of the subject can be seen vertically from the front side of the abdomen), even if the subject were subjected to posture change during the endoscopic examination.

The endoscope insertion position detecting unit 20 can acquire insertion length that represents the length of a part of the endoscope 11 inserted into the large intestine, and elapsed time since the endoscope 11 has been inserted into the large intestine (hereinafter, referred to as an insertion time). The endoscope insertion position detecting unit 20, for example, measures the insertion length from a base point which corresponds to a position at a timing when the operator entered an inspection start operation to the input device 42, and measures the insertion time from a start point which corresponds to the timing. Note that the endoscope insertion position detecting unit 20 may alternatively estimate the position of the anus, from the generated three-dimensional endoscope shape, and from difference in the magnetic field intensity between the magnetic coil inside the body and the magnetic coil outside the body, and may use the estimated position of anus as the base point of the insertion length.

An encoder may be installed near the anus of the subject, for precise measurement of the insertion length. The endoscope insertion position detecting unit 20 detects the insertion length while assuming the position of anus as the base point, with reference to a signal from the encoder.

The endoscope insertion position detecting unit 20 adds the insertion length and the insertion time, to the three-dimensional endoscope shape after the posture correction based on the three-dimensional posture information, and outputs the result to the endoscope insertion assisting system 30.

The endoscope insertion assisting system 30 generates the insertion assisting information of the endoscope 11, with reference to the endoscopic image input from the endoscope system 10, and the endoscope shape input from the endoscope insertion position detecting unit 20, and presents the information to the operator. The endoscope insertion assisting system 30 also generates endoscopic examination history information, with reference to the endoscopic image input from the endoscope system 10, and the endoscope shape input from the endoscope insertion position detecting unit 20, and stores the information in the storage device 43.

The display device 41 has a liquid crystal monitor or an organic EL monitor, on which an image input from the endoscope insertion assisting system 30 is displayed. The input device 42 has a mouse, a keyboard, a touch panel and the like, and outputs operation information entered by an operator or the like to the endoscope insertion assisting system 30. The storage device 43 has a storage medium such as HDD or SSD, and stores the endoscopic examination history information generated by the endoscope insertion assisting system 30. The storage device 43 may be a dedicated storage device attached to the endoscope system 10, may be a database in an in-hospital server connected via an in-hospital network, or may be a database in a cloud server.

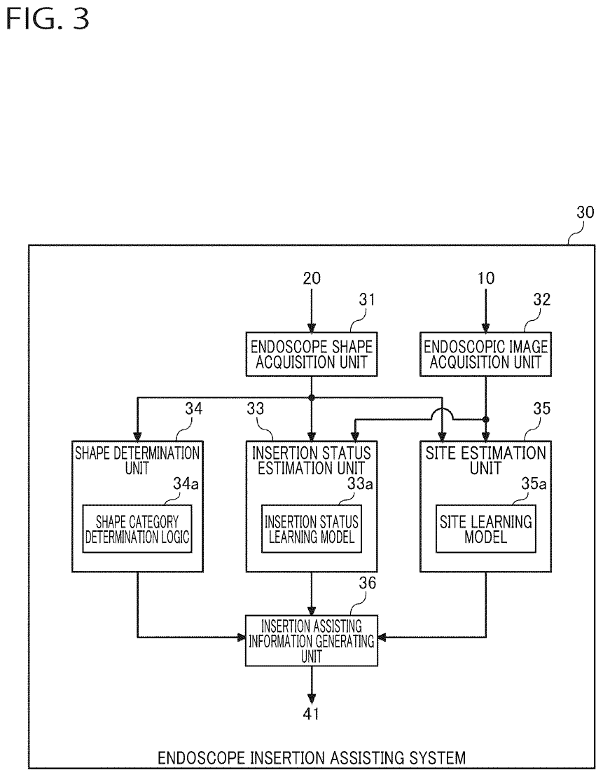

FIG. 3 is a drawing illustrating an exemplary structure of the endoscope insertion assisting system 30 according to First Embodiment. The endoscope insertion assisting system 30 may be constituted by a processing device dedicated to endoscope insertion assistance, or may be constituted by a general-purpose server (may be a cloud server). The endoscope insertion assisting system 30 may also be constituted by a free combination of the processing device dedicated to endoscope insertion assistance, a general-purpose server (may be a cloud server), and a dedicated image diagnosing device. The endoscope insertion assisting system 30 may alternatively be constituted integrally with the endoscope system 10.

The endoscope insertion assisting system 30 includes an endoscope shape acquisition unit 31, an endoscopic image acquisition unit 32, an insertion status estimation unit 33, a shape determination unit 34, a site estimation unit 35, and an insertion assisting information generating unit 36. These components may be implemented by at least one freely selectable processor (such as CPU and GPU), memory (such as DRAM), or other LSI (such as FPGA of ASIC) in terms of hardware, meanwhile may be implemented by a program product loaded in a memory in terms of software. The drawing herein illustrates functional blocks embodied by cooperation of these components. Those skilled in the art will therefore easily understand that these functional blocks can be implemented in various ways, such as solely by hardware, solely by software, or by combination of them.

The endoscope shape acquisition unit 31 acquires the shape information of the insertion part 11a of the endoscope 11 (simply referred to as an endoscope insertion part, hereinafter) during the endoscopic examination, from the endoscope insertion position detecting unit 20. The endoscopic image acquisition unit 32 acquires the endoscopic image during the endoscopic examination, from the endoscope system 10.

The insertion status estimation unit 33 has an insertion status learning model 33a. The insertion status estimation unit 33 enters the shape information of the endoscope insertion part acquired by the endoscope shape acquisition unit 31 to the insertion status learning model 33a, thereby acquiring, from the insertion status learning model 33a, an insertion status category resulted from categorization of the insertion status of the endoscope insertion part. The insertion status of the endoscope insertion part represents an arrangement status of the entire endoscope insertion part in the body of the subject. The insertion status category of the endoscope insertion part represents where and in what form is the endoscope insertion part is arranged in the body of the subject, while dividing the whole of the endoscope insertion part into several steps of insertion, and classifying them into a plurality of deformed presentations.

The insertion status learning model 33a is generated by machine learning with use of, as a supervised data set, a large number of shape information of the endoscope insertion part with the insertion status category of the endoscope insertion part annotated thereto. The annotation is given by an expertized annotator such as doctor. The machine learning can employ CNN, RNN, LSTM or the like, which is a sort of deep learning.

The data set may alternatively be given by a combination of a large number of shape information of the endoscope insertion part and a larger number of endoscopic images, to which the insertion status category of the endoscope insertion part is annotated. The insertion status estimation unit 33 in this case enters the shape information of the endoscope insertion part and the corresponded endoscopic image to the insertion status learning model 33a, thereby acquiring, from the insertion status learning model 33a, the insertion status category.

The site estimation unit 35 has a site learning model 35a. The site estimation unit 35 enters the endoscopic image acquired by the endoscopic image acquisition unit 32 to the site learning model 35a, thereby acquiring, from the site learning model 35a, a site of the subject seen in the endoscopic image. The site learning model 35a is generated by machine learning with use of, as a supervised data set, a large number of endoscopic images with the site of large intestine annotated thereto.

The data set may alternatively be given by a combination of a large number of endoscopic images and a larger number of shape information of the endoscope insertion part, to which the site of large intestine is annotated. The site estimation unit 35 in this case enters the endoscopic images, and corresponded shape information of the endoscope insertion part to the site learning model 35a, thereby acquiring, from the site learning model 35a, the site of large intestine.

The acquired site may be a site where the distal end rigid part 11b of the endoscope 11 (simply referred to as endoscope distal end, hereinafter) resides, or may be sites where a plurality of parts of the endoscope insertion part is individually located. The plurality of parts of the endoscope insertion part may be parts where the magnetic coils 12 are individually arranged. The site of large intestine may be classified into five sites including sigmoid colon, descending colon, transverse colon, ascending colon, and the cecum; may be classified into two sites including sigmoid colon and the site beyond sigmoid colon; or may be classified into any other units.

In a case where the site of the subject to be detected by the site learning model 35a is not detected in time sequence for a predetermined time period, the site estimation unit 35 may invalidate the detected site of the subject. That is, the site estimation unit 35 may assign “delay” to the estimation result of site. The “delay” herein refers to a process of determining misrecognition, in a case where the same estimation result is not returned in a predetermined number of frames, when a site is switched.

In an exemplary case with the frame rate of the endoscopic image set to 60 Hz, and with the “delay” set to 1 second, the site estimation unit 35 determines misrecognition, if the same estimation result is not returned with reference to 60 temporally consecutive frames of the endoscopic image. In another exemplary case with the frame rate of the endoscopic image set to 60 Hz, with the acquisition rate of the shape information of the endoscope insertion part set to 5 Hz, and with the “delay” set to 1 second, the site estimation unit 35 determines misrecognition, if the same estimation result is not returned with reference a combination of 60 temporally consecutive frames of the endoscopic image and corresponded shape information of five endoscope insertion parts.

The shape determination unit 34 has a shape category determination logic 34a. The shape determination unit 34 applies the shape information of the endoscope insertion part acquired by the endoscope shape acquisition unit 31 to the shape category determination logic 34a, thereby acquiring an insertion shape category resulted from categorization of the insertion shape of the endoscope insertion part. The shape category determination logic 34a is a determination logic for geometrically classifying the shape of the endoscope insertion part (typically specified by geometric conditions), and is preliminarily determined by a designer. The shape determination unit 34 can apply, for example, the shape information of the endoscope insertion part to the shape category determination logic 34a, to determine various loops (counterclockwise, N, a (front/back), reverse a (front/back), Y (front/back), re-loop) of the endoscope distal end, and presence of hooking at the central part of the transverse colon.

Hereinafter, features of estimation of the insertion status of the endoscope insertion part by the insertion status estimation unit 33, and determination of the insertion shape of the endoscope insertion part by the shape determination unit 34 will be compared. The estimation by the insertion status estimation unit 33 has wide allowance for recognition of the insertion status. That is, the insertion status can be categorized to an insertion status category with the highest similarity, even if the insertion status is not completely matched to any of the insertion status categories. This method also enables estimation of the insertion status of the endoscope insertion part, also involving the site. The method can also use time series information. Sites of large intestine are arranged in the order, from the anal side, of sigmoid colon, descending colon, transverse colon, ascending colon, and cecum. The estimation by the insertion status estimation unit 33 can also specify the site with use of the time series information. Note, however, that estimation accuracy by the insertion status estimation unit 33 depends on the data set to be learned. The estimation accuracy varies depending on the insertion status category.

On the other hand, the determination by the shape determination unit 34, can recognize, with high accuracy, the shape set by the determination logic. Note, however, that the determination by the shape determination unit 34 has less allowance for recognition of the insertion shape. That is, the shape cannot be recognized even under tiny deviation from the set conditions. The determination by the shape determination unit 34, without use of the learning data set, thus suffers from limitation of conditions that can be set.

It now becomes possible to reduce misrecognition due to variation in the accuracy of the learning data set, by combining the estimation result given by the insertion status estimation unit 33 and the determination result given by the shape determination unit 34, while enabling wide allowance of recognition including the site, obtainable only from the shape information of the endoscope insertion part. For example, the recognition accuracy may be improved by correcting the estimation result of the insertion status category by the insertion status estimation unit 33 derived from an insufficient amount of learning data set, with the determination result given by the shape determination unit 34.

The misrecognition would, however, be still likely to occur in a case with similar insertion shape but at different site, only with the aid of the estimation of the insertion status by the insertion status estimation unit 33 and the determination of the insertion shape by the shape determination unit 34. Although the “delay” might be set also to the estimation of the insertion status by the insertion status estimation unit 33 similarly to the site estimation unit 35, it would be difficult to set a long “delay” for the estimation of the insertion status by the insertion status estimation unit 33. The long “delay”, during which the insertion shape would vary, will fail to obtain the same result. The “delay” will therefore be less effective to prevent misrecognition, even if set to the estimation by the insertion status estimation unit 33.

Now, by combining the estimation result of site by the site estimation unit 35, having been provided with “delay” so as to reduce the misrecognition, with the estimation result of the insertion status by the insertion status estimation unit 33 and with the determination result of the insertion shape by the shape determination unit 34, it becomes possible to more correctly determine in what insertion shape and where the endoscope insertion part resides. That is, with the insertion status category including the site estimated by the insertion status estimation unit 33; with the correctness of the insertion shape which can be poorly estimated by the insertion status estimation unit 33 complemented by the shape determination unit 34; and with the correctness of the site which can be poorly estimated by the insertion status estimation unit 33 complemented by the site estimation unit 35, it now becomes possible to more correctly recognize almost all the insertion statuses that can be estimated by the insertion status estimation unit 33.

The insertion assisting information generating unit 36 generates the insertion assisting information, with reference to the insertion status category. The insertion assisting information generating unit 36 outputs the thus generated insertion assisting information to the display device 41, to be displayed thereon. The insertion assisting information includes operation assisting information of the endoscope 11 for the operator. The insertion assisting information is preliminarily linked to each of the insertion status categories.

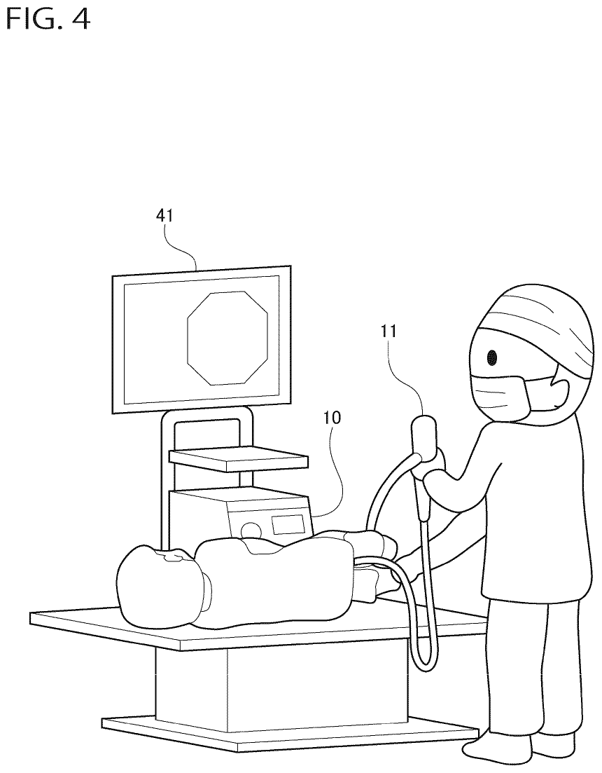

FIG. 4 is a drawing illustrating a scene of colonoscopy. The operator inserts the endoscope 11 into the anus of the subject for endoscopic examination, while monitoring the endoscopic image captured by the endoscope 11 and the insertion assisting information displayed on the display device 41.

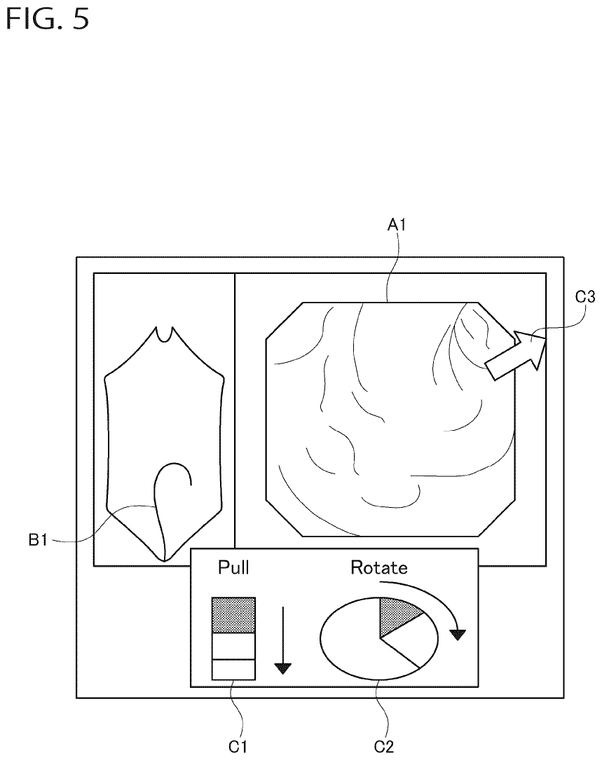

FIG. 5 is a diagram illustrating an exemplary screen that appears on the display device 41 during the endoscopic examination. The screen contains an endoscopic image A1 captured by the endoscope 11 displayed on the right, and contains an endoscope shape B1 displayed on the left. A guide C3 that indicates a travel direction of the endoscope 11 is displayed on the upper right of the endoscopic image A1; and a guide C1 for recommending a pull operation and a guide C2 for recommending a right torque operation are displayed as the operation assisting information, below the endoscopic image A1.

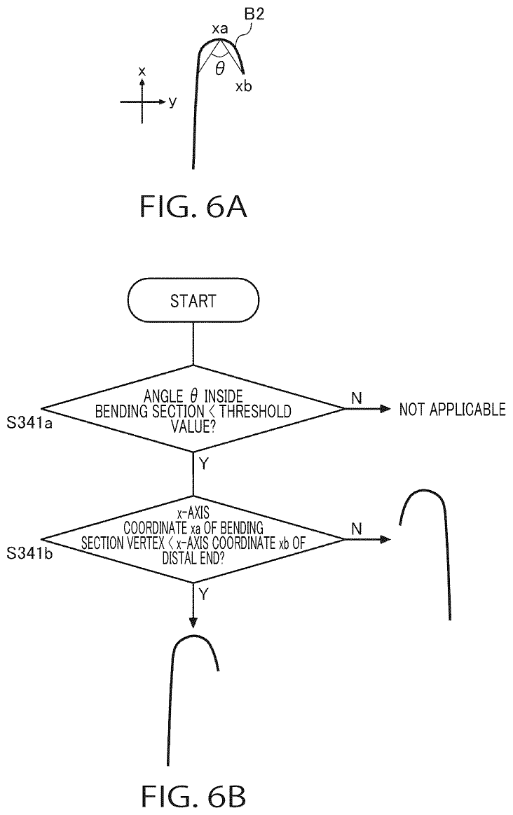

FIGS. 6A and 6B are drawings explaining an exemplary determination logic used in the shape determination unit 34. An Example in FIGS. 6A and 6B illustrates a determination logic for determining the endoscope shape B2 that curves rightward or leftward. FIG. 6A is a drawing illustrating parameters of the endoscope shape B2, and FIG. 6B is a diagram illustrating a conditional expression of the determination logic. The shape determination unit 34 compares an angle θ inside the bending section of the endoscope shape B2 with a threshold value (S341a). If the angle θ inside the bending section is equal to or larger than the threshold (N in S341a), the shape determination unit 34 determines that the endoscope shape B2 does not apply to the determination logic.

If the angle θ inside the bending section is smaller than the threshold (Y in S341a), the shape determination unit 34 compares an x-axis coordinate xa of the vertex of the bending section, with an x-axis coordinate xb of the distal end of the bending section, in the endoscope shape B2 (S341b). If the x-axis coordinate xa of the vertex of the bending section is smaller than the x-axis coordinate xb of the distal end of the bending section (Y in S341b), the shape determination unit 34 determines that the endoscope shape is curved rightward. If the x-axis coordinate xa of the vertex of the bending section is larger than the x-axis coordinate xb of the distal end of the bending section (N in S341b), the shape determination unit 34 determines that the endoscope shape is curved leftward.

In a case where the insertion shape category contained in the insertion status category output from the insertion status estimation unit 33 was found to disagree with the insertion shape category output from the shape determination unit 34, the insertion assisting information generating unit 36 corrects the insertion shape category contained in the insertion status category output from the insertion status estimation unit 33, to the insertion shape category output from the shape determination unit 34.

In a case where the site of the subject contained in the insertion status category output from the insertion status estimation unit 33 was found to disagree with the site of the subject output from the site estimation unit 35, the insertion assisting information generating unit 36 corrects the site of the subject contained in the insertion status category output from the insertion status estimation unit 33, to the site of the subject output from the site estimation unit 35.

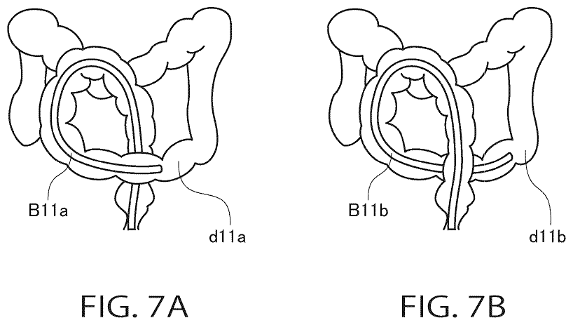

FIGS. 7A and 7B are drawings explaining Correction Example 1 of the insertion status category. FIG. 7A illustrates an actual insertion status of the endoscope insertion part, and FIG. 7B illustrates an insertion status of the endoscope insertion part estimated by the insertion status estimation unit 33. In the actual insertion status (correct) as illustrated in FIG. 7A, the insertion shape B11a of the endoscope insertion part is “front α loop”, and the site d11a where the endoscope distal end resides is “sigmoid colon”. In the insertion status (incorrect) estimated by the insertion status estimation unit 33 as illustrated in FIG. 7B, the insertion shape B11b of the endoscope insertion part is “back α loop”, and the site d11b where the endoscope distal end resides is “sigmoid colon”.

The site where the endoscope distal end resides as estimated by the site estimation unit 35 is “sigmoid colon”. The insertion shape of the endoscope insertion part determined by the shape determination unit 34 is “front α loop”. The site where the endoscope distal end resides, as estimated by the insertion status estimation unit 33, agrees with the site where the endoscope distal end resides, as estimated by the site estimation unit 35, both indicating “sigmoid colon”. The insertion assisting information generating unit 36 therefore determines “sigmoid colon” as the site where the endoscope distal end resides.

The “back α loop”, estimated as the insertion shape of the endoscope insertion part by the insertion status estimation unit 33, is different from the “front α loop”, estimated as the insertion shape of the endoscope insertion part determined by the shape determination unit 34. Since the shape determination unit 34 is superior in terms of recognition accuracy of the loop shape, so that the insertion assisting information generating unit 36 determines the “back α loop”, estimated by the insertion status estimation unit 33, as being misrecognized, instead adopting the “front α loop” determined by the shape determination unit 34.

That is, the insertion assisting information generating unit 36 corrects the insertion shape category, defined by “sigmoid colon” and “back α loop” estimated by the insertion status estimation unit 33, to the insertion shape category defined by “sigmoid colon” and “front α loop”. The insertion assisting information generating unit 36 acquires the insertion assisting information linked to the corrected insertion status category, and displays an operation guide derived from the thus acquired insertion assisting information, on the display device 41.

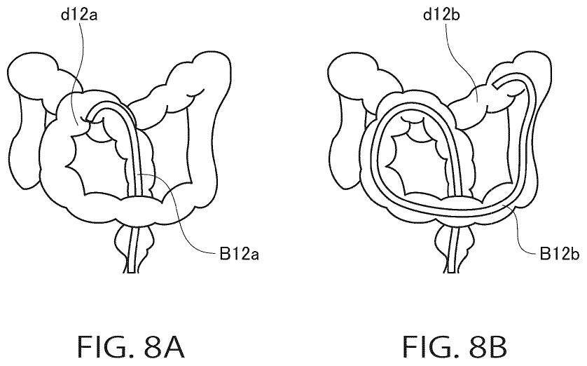

FIGS. 8A and 8B are drawings explaining Correction Example 2 of the insertion status category. FIG. 8A illustrates an actual insertion status of the endoscope insertion part, and FIG. 8B illustrates an insertion status of the endoscope insertion part estimated by the insertion status estimation unit 33. In the actual insertion status (correct) as illustrated in FIG. 8A, the insertion shape B12a of the endoscope insertion part is “no loop, distal end bent leftward”, and the site d12a where the endoscope distal end resides is “sigmoid colon”. In the insertion status (incorrect) estimated by the insertion status estimation unit 33 as illustrated in FIG. 8B, the insertion shape B12b of the endoscope insertion part is “no loop, distal end bent leftward”, and the site d12b where the endoscope distal end resides is “transverse colon (or “splenic flexure” in more detail)”.

The site where the endoscope distal end resides as estimated by the site estimation unit 35 is “sigmoid colon”. The insertion shape of the endoscope insertion part determined by the shape determination unit 34 is “no loop, distal end bent leftward”. “Transverse colon”, which is the site where the endoscope distal end resides as estimated by the insertion status estimation unit 33, is different from “sigmoid colon”, which is the site where the endoscope distal end resides as estimated by the site estimation unit 35. Since the site estimation unit 35 is superior in terms of recognition accuracy of site, so that the insertion assisting information generating unit 36 determines the “transverse colon”, estimated by the insertion status estimation unit 33, as being misrecognized, instead adopting the “sigmoid colon” estimated by the site estimation unit 35.

The insertion shape of the endoscope insertion part, estimated by the insertion status estimation unit 33, agrees with the insertion shape of the endoscope insertion part determined by the shape determination unit 34, both indicating “no loop, distal end bent leftward”. The insertion assisting information generating unit 36 therefore determines “no loop, distal end bent leftward” as the insertion shape of the endoscope insertion part.

That is, the insertion assisting information generating unit 36 corrects the insertion shape category, defined by “transverse colon” and “no loop, distal end bent leftward” estimated by the insertion status estimation unit 33, to the insertion shape category defined by “sigmoid colon” and “no loop, distal end bent leftward”.

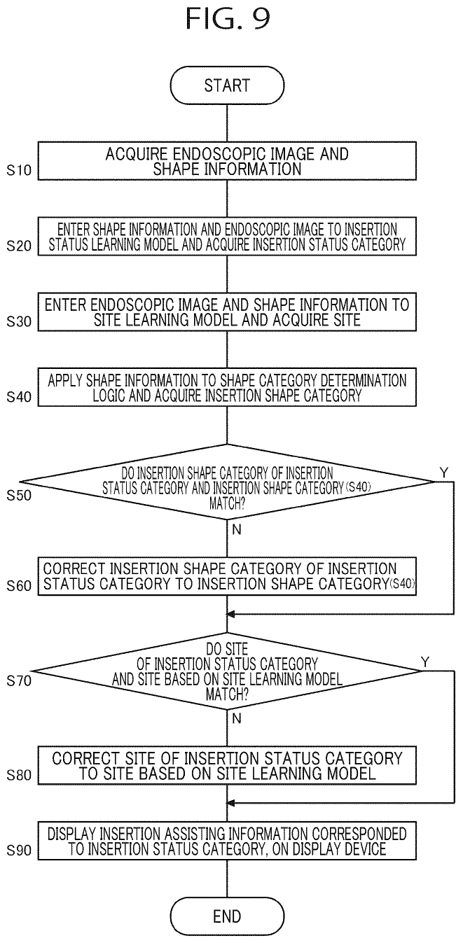

FIG. 9 is a flow chart illustrating an exemplary operation of the endoscope insertion assisting system 30 according to First Embodiment. The endoscopic image acquisition unit 32 acquires the endoscopic image from the endoscope system 10, and the endoscope shape acquisition unit 31 acquires the shape information of the endoscope insertion part from the endoscope insertion position detecting unit 20 (S10).

The insertion status estimation unit 33 enters the shape information of the endoscope insertion part and the endoscopic image to the insertion status learning model 33a, thereby acquiring, from the insertion status learning model 33a, the insertion status category of the endoscope insertion part (S20). The site estimation unit 35 enters the endoscopic images and the shape information of the endoscope insertion part to the site learning model 35a, thereby acquiring, from the site learning model 35a, the site where the endoscope distal end resides (S30). The shape determination unit 34 applies the shape information of the endoscope insertion part to the shape category determination logic 34a, thereby acquiring, from the shape category determination logic 34a, the insertion shape category (S40). The processes of steps S20, S30, and S40 may be executed sequentially, or in parallel.

The insertion assisting information generating unit 36 compares the insertion shape category contained in the insertion status category estimated by the insertion status estimation unit 33, with the insertion shape category determined by the shape determination unit 34 (S50). If these categories do not match (N in S50), the insertion assisting information generating unit 36 corrects the insertion shape category contained in the insertion status category estimated by the insertion status estimation unit 33, to the insertion shape category determined by the shape determination unit 34 (S60). If these categories match (Y in S50), the process in step S60 is skipped.

The insertion assisting information generating unit 36 compares the site where the endoscope distal end contained in the insertion status category resides, as estimated by the insertion status estimation unit 33, with the site where the endoscope distal end resides as estimated by the site estimation unit 35 (S70). If these sites do not match (N in S70), the insertion assisting information generating unit 36 corrects the site where the endoscope distal end contained in the insertion status category resides, as estimated by the insertion status estimation unit 33, to the site where the endoscope distal end resides, as estimated by the site estimation unit 35 (S80). If these sites match (Y in S70), the process in step S80 is skipped. The insertion assisting information generating unit 36 acquires the insertion assisting information linked to the finally determined insertion status category, and displays the thus acquired insertion assisting information, on the display device 41 (S90).

Note that priority information, specifying which of the insertion shape category contained in the insertion status category estimated by the insertion status estimation unit 33, or, the insertion shape category determined by the shape determination unit 34 has priority, may be preliminarily attached to each of the plurality of insertion status categories having been prepared as the estimation result of the insertion status learning model 33a.

Alternatively, priority information, specifying which of the insertion shape category contained in the insertion status category estimated by the insertion status estimation unit 33, or, the insertion shape category determined by the shape determination unit 34 has priority, may be preliminarily attached to each of the plurality of insertion shape categories having been prepared as the determination result of the shape category determination logic 34a.

The insertion assisting information generating unit 36 determines whether or not to correct the insertion shape category contained in the insertion status category acquired from the insertion status learning model 33a, to the insertion shape category acquired from the shape category determination logic 34a, with reference to the priority information assigned to at least either the insertion shape category contained in the insertion status category estimated by the insertion status estimation unit 33, or the insertion shape category determined by the shape determination unit 34.

If the insertion shape category contained in the insertion status category estimated by the insertion status estimation unit 33 has higher priority, the insertion assisting information generating unit 36 does not correct the insertion shape category contained in the insertion status category acquired from the insertion status learning model 33a. In contrast, if the insertion shape category determined by the shape determination unit 34 has higher priority, the insertion assisting information generating unit 36 corrects the insertion shape category contained in the insertion status category acquired from the insertion status learning model 33a, to the insertion shape category acquired from the shape category determination logic 34a.

When entering the shape information of the endoscope insertion part (or combination of the shape information and the endoscopic image) to the insertion status learning model 33a to acquire the insertion status category, the insertion status estimation unit 33 can acquire, at the same time, reliability based on similarity between the entered shape information (or combination of the shape information and the endoscopic image) and the classified insertion status category.

The insertion assisting information generating unit 36 can determine whether or not to correct the insertion shape category contained in the insertion status category acquired from the insertion status learning model 33a, to the insertion shape category acquired from the shape category determination logic 34a, with reference to the reliability of the insertion status category acquired from the insertion status learning model 33a. If the reliability is higher than a threshold value, the insertion assisting information generating unit 36 does not correct the insertion shape category contained in the insertion status category acquired from the insertion status learning model 33a. In contrast, if the reliability is smaller than the threshold value, the insertion assisting information generating unit 36 corrects the insertion shape category contained in the insertion status category acquired from the insertion status learning model 33a, to the insertion shape category acquired from the shape category determination logic 34a.

As described above, the determination by the shape determination unit 34 may encounter a case where the shape information of the endoscope insertion part satisfies none of the conditions of the shape category determination logic 34a. In a case where the shape information of the endoscope insertion part satisfies none of the conditions of the shape category determination logic 34a, and the reliability of the insertion status category acquired from the insertion status learning model 33a is lower than the threshold value, the insertion assisting information generating unit 36 determines that the insertion shape of the endoscope insertion part cannot be estimated.

The insertion assisting information generating unit 36 does not display an operation guide based on the insertion assisting information on the display device 41, during a period over which the insertion shape of the endoscope insertion part cannot be estimated. The insertion assisting information generating unit 36 may present a message such as “Insertion status of endoscope not recognizable. Operate with care.”, on the display device 41.

The description have been made on the premise that all of the insertion status estimation process by the insertion status estimation unit 33, the insertion shape determination process by the shape determination unit 34, and the site estimation process by the site estimation unit 35 take place. In this respect, it is alternatively possible to skip either the insertion status estimation process by the insertion status estimation unit 33, or the insertion shape determination process by the shape determination unit 34, according to a predetermined condition. The predetermined condition may be determined according to a result of priority determination that determines which output from the insertion status estimation unit 33 or the shape determination unit 34 is to be prioritized.

For example, the insertion status estimation process by the insertion status estimation unit 33 may precede the insertion shape determination process by the shape determination unit 34. If the reliability of the insertion status category acquired from the insertion status learning model 33a is higher than the threshold value, the insertion shape determination process by the shape determination unit 34 is omissible. The insertion assisting information generating unit 36 in this case adopts the insertion status category acquired from the insertion status learning model 33a as it is.

For example, the insertion shape determination process by the shape determination unit 34 may precede the insertion status estimation process by the insertion status estimation unit 33. If the insertion shape category was successfully acquired from the shape category determination logic 34a, the insertion status estimation process by the insertion status estimation unit 33 is omissible. The insertion assisting information generating unit 36 in this case specifies the insertion shape category acquired from the shape category determination logic 34a, and the insertion status category containing the site acquired from the site learning model 35a.

Which of the insertion status estimation process by the insertion status estimation unit 33, and the insertion shape determination process by the shape determination unit 34 is to precede, may be determined with reference to the latest adoption rates of the insertion shape category contained in the insertion status category acquired from the insertion status learning model 33a, and the insertion shape category acquired from the shape category determination logic 34a. For example, in a case where the latest adoption rate of the insertion shape category acquired from the shape category determination logic 34a is higher, the insertion shape determination process by the shape determination unit 34 precedes.

In this example, it is not always necessary to preliminarily attach the priority information, to each insertion status category having been prepared as the estimation result given by the insertion status learning model 33a, or to each insertion shape category having been prepared as the determination result given by the shape category determination logic 34a. The process can take place, if only the shape information of the endoscope insertion part is entered either to the insertion status estimation unit 33, or to the shape determination unit 34.

It is also acceptable to provide a priority determination unit that determines which output of the insertion status estimation unit 33 or the shape determination unit 34 is to be prioritized, with reference to the shape information of the endoscope insertion part acquired by the endoscope shape acquisition unit 31. The insertion assisting information generating unit 36 in this case may only acquire the output of insertion shape category either from the insertion status estimation unit 33 or from the shape determination unit 34, whichever is given priority. In a case where the shape determination unit 34 is prioritized, the insertion assisting information generating unit 36 can use a combination of the insertion shape category information from the shape determination unit 34, and the information of the site where the endoscope distal end resides from the site estimation unit 35. That is, the output from the non-prioritized one of the insertion status estimation unit 33 or the shape determination unit 34 is not essential.

As has been described, First Embodiment can reduce misrecognition of the insertion status of the endoscope, by combining the estimation result given by the insertion status estimation unit 33, with at least either the determination result given by the shape determination unit 34, or the estimation result of a site given by the site estimation unit 35. For an exemplary insertion shape that AI tends to misrecognize, misrecognition of the insertion shape may be reduced by adopting the insertion shape determined according to a determination logic set by the designer.

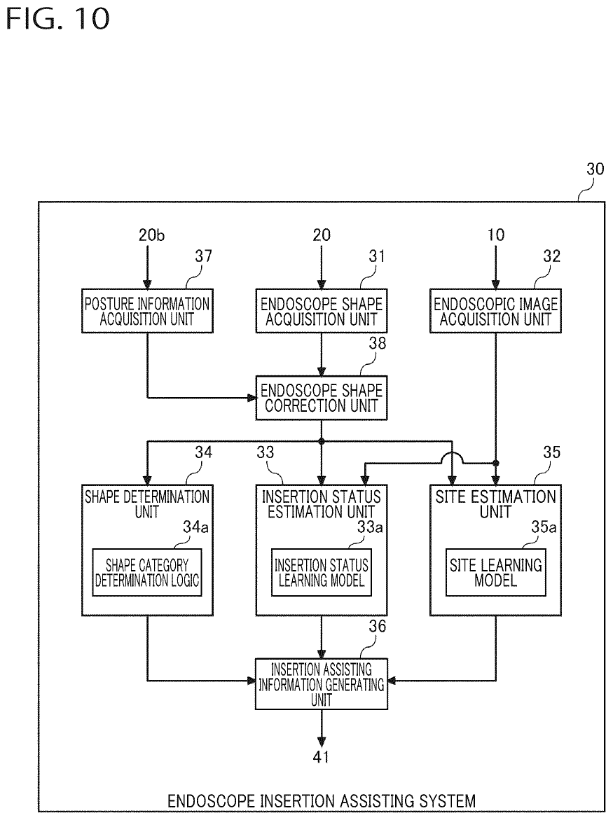

FIG. 10 is a drawing illustrating an exemplary structure of the endoscope insertion assisting system 30 according to Second Embodiment. An endoscope insertion assisting system 30 according to Second Embodiment is structured to have a posture information acquisition unit 37 and an endoscope shape correction unit 38, which are added to the structure of the endoscope insertion assisting system 30 according to First Embodiment illustrated in FIG. 3.

The description regarding FIG. 1 has dealt with a case where the reference plate 20b having the posture sensor arranged thereon is connected to the endoscope insertion position detecting unit 20. Second Embodiment now assumes an exemplary case where the reference plate 20b having the posture sensor arranged thereon is directly connected to the endoscope insertion assisting system 30. The shape information used in the insertion status estimation unit 33, the shape determination unit 34, and the site estimation unit 35 stand on the viewpoint from the abdominal side of the subject in the supine position. Any change in posture of the subject during examination would therefore cause misrecognition of the endoscope shape differently from the actual one.

The posture information acquisition unit 37 acquires, from the reference plate 20b, three-dimensional information that indicates a posture of the subject as the posture information. Note that Second Embodiment is not limited to an example in which the posture sensor is attached to the subject to measure the posture of the subject. For example, a plurality of pressure sensors may be mounted on an examination bed, and the posture of the subject may be estimated with reference to the detected values from the individual pressure sensors. Alternatively, a camera may be installed in an examination room, and movement of the subject that appears in a movie captured by the camera may be tracked by image recognition, to estimate the posture of the subject.

The endoscope shape correction unit 38 corrects the shape information of the endoscope insertion part acquired by the endoscope shape acquisition unit 31, with reference to the posture information acquired by the posture information acquisition unit 37. More specifically, the endoscope shape correction unit 38 changes the shape information of the endoscope insertion part, so as to cancel a three-dimensional change in the posture of the subject defined by the posture information.

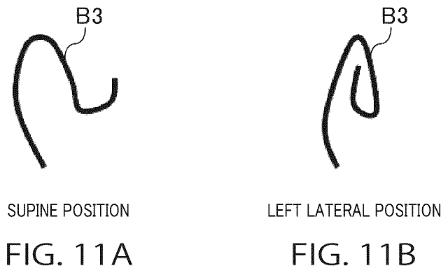

FIGS. 11A and 11B are drawing illustrating exemplary endoscope shapes B3 before and after posture change. FIG. 11A illustrates an exemplary ceiling view of an endoscope shape B3 inserted into the subject in the supine position. FIG. 11B illustrates an exemplary ceiling view of an endoscope shape B3 inserted into the subject in the left lateral position. Change in the posture of the subject largely changes the ceiling view of the endoscope shape B3.

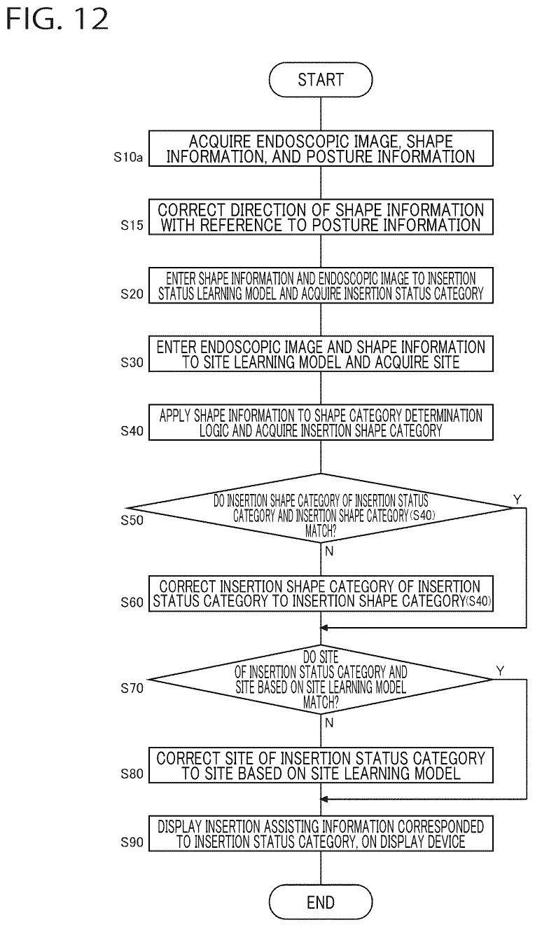

FIG. 12 is a flow chart illustrating an exemplary operation of the endoscope insertion assisting system 30 according to Second Embodiment. The endoscopic image acquisition unit 32 acquires the endoscopic image from the endoscope system 10, the endoscope shape acquisition unit 31 acquires the shape information of the endoscope insertion part from the endoscope insertion position detecting unit 20, and the posture information acquisition unit 37 acquires the posture information from the posture sensor (S10a).

The endoscope shape correction unit 38 corrects the direction of the acquired shape information with reference to the posture information. For example, the endoscope shape correction unit 38 always correct that the acquired shape information will be the shape information viewed from the abdominal side of the subject (S15). Processes in step S20 and thereafter are same as those illustrated in the flow chart of FIG. 9.

As described above, Second Embodiment has the following effects, besides the effects of First Embodiment. In Second Embodiment, acquisition of the posture information enables to grasp in what position the subject is laid now. By correcting the shape information of the endoscope insertion part with reference to the posture information, the shape information may always be handled as viewed from the abdominal side of the subject.

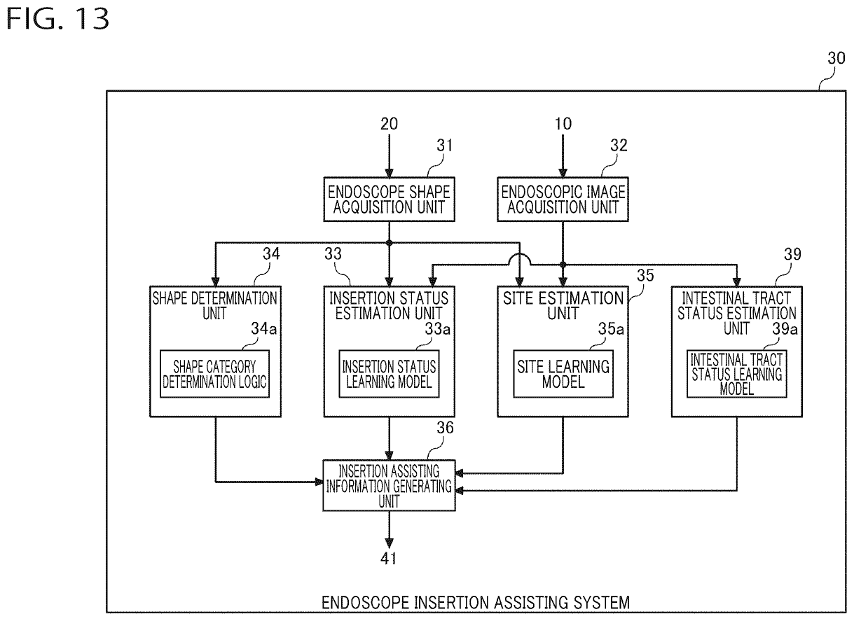

FIG. 13 is a drawing illustrating an exemplary structure of an endoscope insertion assisting system 30 according to Third Embodiment. The endoscope insertion assisting system 30 according to Third Embodiment is structured to have an intestinal tract status estimation unit 39, which is added to the structure of the endoscope insertion assisting system 30 according to First Embodiment illustrated in FIG. 3.

The intestinal tract status estimation unit 39 enters the endoscopic image acquired by the endoscopic image acquisition unit 32 to an intestinal tract status learning model 39a, thereby acquiring, from the intestinal tract status learning model 39a, a status regarding a distance between the endoscope distal end and the intestinal wall of the subject (referred to as intestinal wall distance status, hereinafter). The intestinal tract status estimation unit 39 can typically acquire any of “optimal distance”, “straight pipe” or “red ball” as the intestinal wall distance status. “Optimal distance” refers to a status in which the distance between the endoscope distal end and the intestinal wall falls within a predetermined range. “Straight pipe” refers to a status in which the lumen extends straight with good visibility. “Red ball” refers to a status in which the endoscope distal end comes into contact with or approaches the intestinal wall, giving a bright red view.

The intestinal tract status estimation unit 39 may also enter the endoscopic image acquired by the endoscopic image acquisition unit 32 to an intestinal tract status learning model 39a, thereby acquiring, from the intestinal tract status learning model 39a, a status regarding a residue or water. That is, the intestinal tract status estimation unit 39 can specify the endoscopic image having the residue or water captured therein. The intestinal tract status estimation unit 39 may also be able to specify an endoscopic image with cleaning liquid, bleeding, diverticulum, or lesion (polyp or the like) captured therein, besides the residue or water.

The intestinal tract status learning model 39a is generated by machine learning with use of, as a supervised data set, the intestinal wall distance status, and a large number of endoscopic images annotated with information as to whether the residue, water, washing liquid, bleeding, diverticulum, or lesion is captured or not.

The insertion assisting information generating unit 36 generates the insertion assisting information with reference to the insertion status category and the intestinal wall distance status, and displays it on the display device 41. With reference to the intestinal wall distance status, the insertion assisting information generating unit 36 can grasp the insertion status in further detail, and can present a more detailed insertion assisting information suitable for the insertion status.

In an exemplary case where the intestinal wall distance status has not been acquired, in the insertion status category in which the site where the endoscope distal end resides is defined by “sigmoid colon (more specifically, near the first curve on the rectum side of sigmoid colon)”, and the insertion shape of the endoscope insertion part is defined by “no loop, distal end bent rightward”, the insertion assisting information generating unit 36 generates a guide that recommends right torque operation as the insertion assisting information. Also in a case where the intestinal wall distance state has been acquired, and the intestinal wall distance status is given by “optimal distance”, the insertion assisting information generating unit 36 similarly generates a guide that recommends right torque operation as the insertion assisting information. In contrast, in a case where the intestinal wall distance state is defined by “straight pipe”, the insertion assisting information generating unit 36 generates, as the insertion assisting information, a guide that recommends pull operation, or operation of bringing the endoscope distal end close to the intestinal wall by suction.

The insertion assisting information generating unit 36 can also generate the insertion assisting information, with reference to the insertion status category, and the status regarding the residue or water. In a case where the endoscopic image has the residue or water captured therein, the insertion assisting information generating unit 36 generates a guide that recommends cleaning or suction, for example.

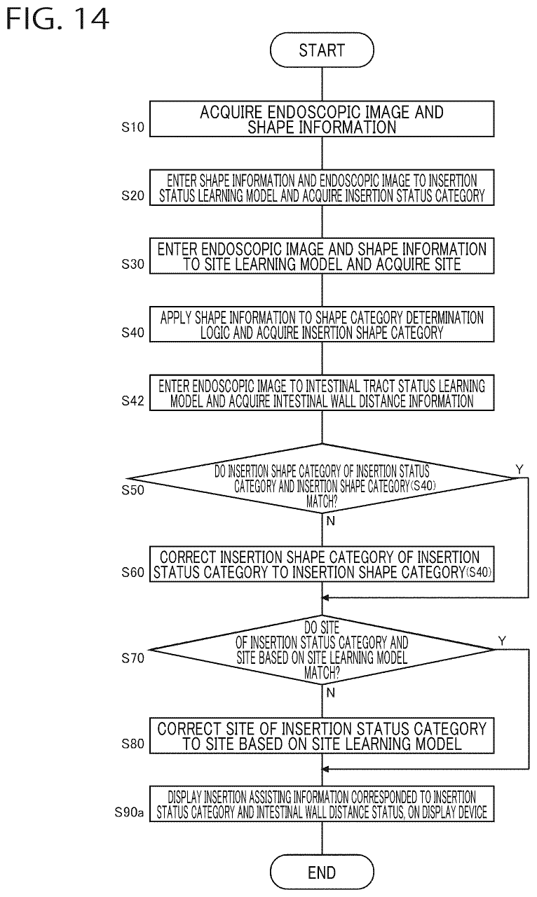

FIG. 14 is a flow chart illustrating an exemplary operation of the endoscope insertion assisting system 30 according to Third Embodiment. Processes in step S10 to step S40 are same as those illustrated in the flow chart of FIG. 9. The intestinal tract status estimation unit 39 enters the endoscopic image acquired by the endoscopic image acquisition unit 32 to the intestinal tract status learning model 39a, thereby acquiring, from the intestinal tract status learning model 39a, the intestinal wall distance status (S42). Processes in step S50 to step S80 are same as those illustrated in the flow chart of FIG. 9. The insertion assisting information generating unit 36 acquires the insertion assisting information linked to a combination of the finally determined insertion status category and the intestinal wall distance status, and displays the thus acquired insertion assisting information on the display device 41 (S90a).

Note that the insertion status learning model 33a may be generated by machine learning with use of, as a supervised data set, a combination of a large number of shape information of the endoscope insertion part with the insertion status category of the endoscope insertion part annotated thereto, and a large number of endoscopic images (with intestinal wall distance status). The insertion status estimation unit 33 in this case enters the shape information of the endoscope insertion part, the corresponded endoscopic image, and the corresponded intestinal wall distance status to the insertion status learning model 33a, thereby acquiring, from the insertion status learning model 33a, the insertion status category.

As described above, Third Embodiment has the following effects, besides the effects of First Embodiment. With reference to the intestinal tract status, Third Embodiment can present a more detailed insertion assisting information.

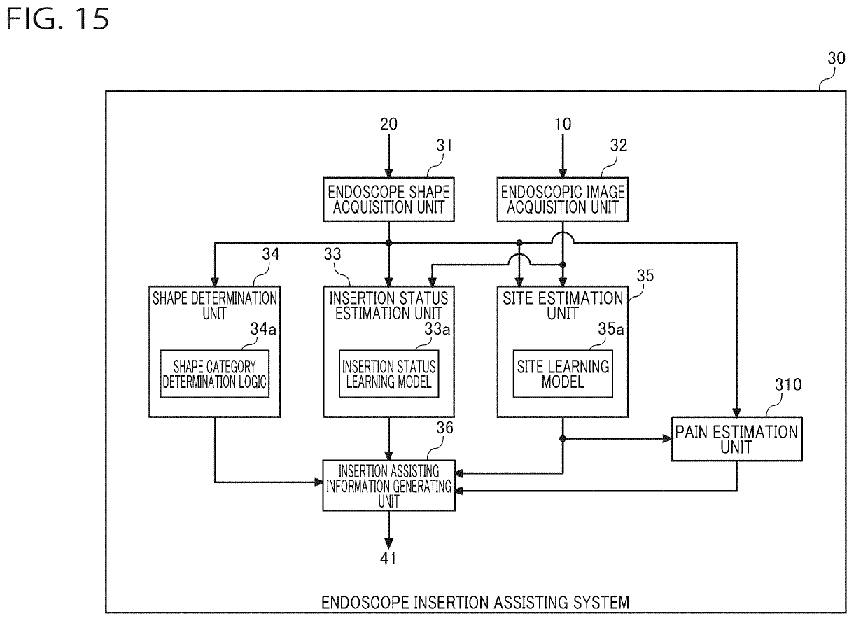

FIG. 15 illustrates an exemplary structure of the endoscope insertion assisting system 30 according to Fourth Embodiment. The endoscope insertion assisting system 30 according to Fourth Embodiment is structured to have a pain estimation unit 310, which is added to the structure of the endoscope insertion assisting system 30 according to First Embodiment illustrated in FIG. 3.

The pain estimation unit 310 generates pain information that indicates pain occurred in the subject, with reference to the insertion shape of the endoscope insertion part, and the site where the endoscope distal end resides. The pain information contains at least the presence or absence of pain. Degree of pain may also be contained. A table is preliminarily prepared, in which the pain information is specified for each combination of the insertion shape of the endoscope insertion part and the site where the endoscope distal end resides. The site where the endoscope distal end resides usable herein may be the site where the endoscope distal end resides, estimated by the site estimation unit 35.

The insertion shape of the endoscope insertion part usable herein may be the insertion shape category determined by the shape determination unit 34, or may be an insertion shape classified by a category classification different from the category classification for the insertion shape by the shape determination unit 34. In the latter case, an insertion shape learning model for pain estimation, or an insertion shape determination logic for pain estimation is separately prepared.

The insertion assisting information generating unit 36 generates the insertion assisting information, with reference to the insertion status category and the pain information, and displays the insertion assisting information on the display device 41. With reference to the pain information, it now becomes possible to present insertion assisting information unique to occurrence of pain.

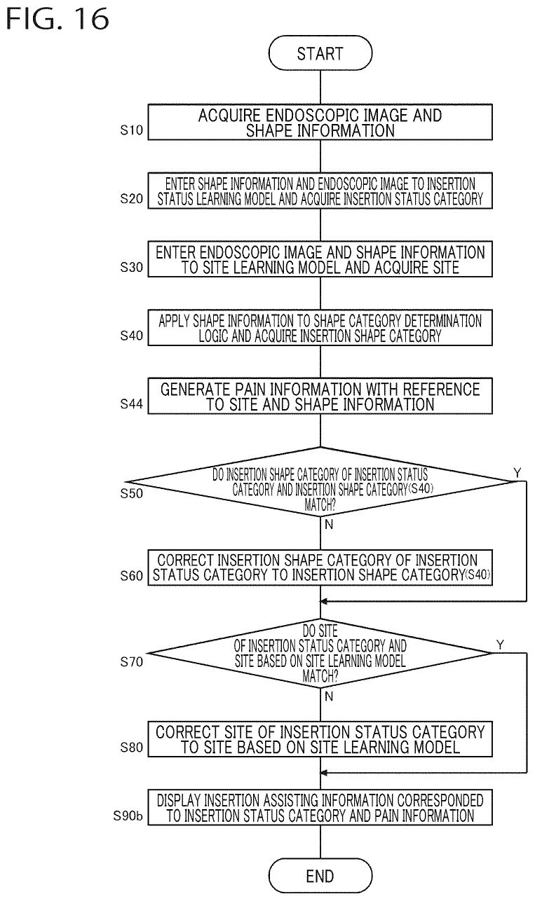

FIG. 16 is a flow chart illustrating an exemplary operation of the endoscope insertion assisting system 30 according to Fourth Embodiment. Processes in step S10 to step S40 are same as those illustrated in the flow chart of FIG. 9. The pain estimation unit 310 generates the pain information, with reference to the shape information of the endoscope insertion part acquired by the endoscope shape acquisition unit 31, and the site where the endoscope distal end resides, estimated by the site estimation unit 35 (S44). Processes in step S50 to step S80 are same as those illustrated in the flow chart of FIG. 9. The insertion assisting information generating unit 36 acquires the insertion assisting information with reference to the finally determined insertion status category and the pain information, and displays the thus generated insertion assisting information on the display device 41 (S90b).

As described above, Fourth Embodiment has the following effects, besides the effects of First Embodiment. With reference to the pain information, Fourth Embodiment can present a more detailed insertion assisting information.

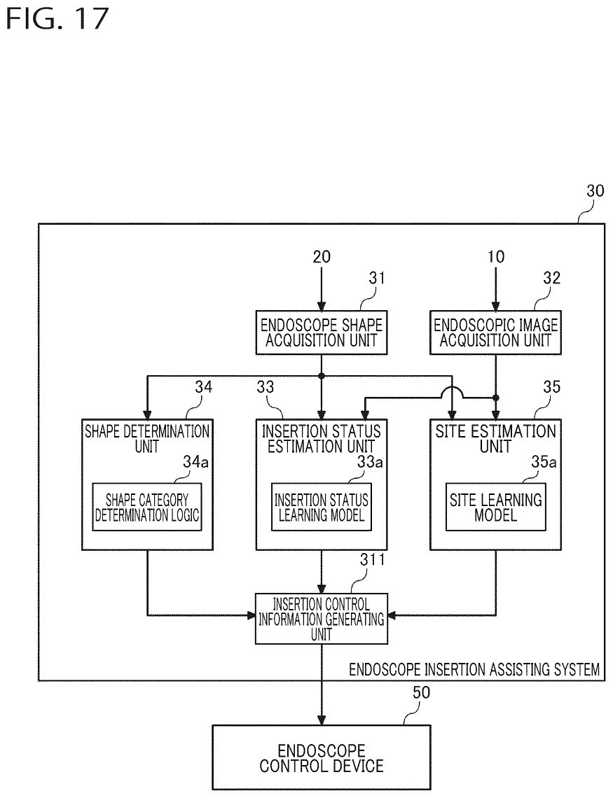

FIG. 17 illustrates an exemplary structure of the endoscope insertion assisting system 30 according to Fifth Embodiment. The endoscope insertion assisting system 30 according to Fifth Embodiment is structured to have an insertion control information generating unit 311, which replaces the insertion assisting information generating unit 36 of the endoscope insertion assisting system 30 according to First Embodiment illustrated in FIG. 3.

The insertion control information generating unit 311 generates insertion control information with reference to the insertion status category, and transmits the thus generated insertion control information to an endoscope control device 50. The endoscope control device 50 is typically a fully automatic or semi-automatic endoscope operating robot. The endoscope control device 50 executes the insertion operation of the endoscope insertion part fully automatically or semi-automatically, with reference to the insertion control information received from the endoscope insertion assisting system 30.

As described above, Fifth Embodiment can reduce possibility of transmitting an erroneous operation instruction to the endoscope control device 50.

The present disclosure has been explained referring to the plurality of embodiments. It is to be understood by those skilled in the art that these embodiments are merely illustrative, that the individual constituents or combinations of various processes may be modified in various ways, and that also such modifications fall within the scope of the present disclosure.

Also in Second to Fifth Embodiments, it is alternatively possible to skip either one of the insertion status estimation process by the insertion status estimation unit 33, or the insertion shape determination process by the shape determination unit 34, according to a predetermined condition, similarly as in First Embodiment. The predetermined condition may be determined according to a result of priority determination that determines which output from the insertion status estimation unit 33 or the shape determination unit 34 is to be prioritized.

Aforementioned Embodiments have dealt with the cases where a plurality of magnetic coils are embedded in the endoscope 11 to estimate the endoscope shape. In this regard, a plurality of shape sensors may alternatively be embedded in the endoscope 11 to estimate the endoscope shape. The shape sensor may be, for example, a fiber sensor that detects a bent shape from a curvature of a specific site, with use of an optical fiber. The fiber sensor typically has an optical fiber laid in the longitudinal direction of the insertion part 11a, and a plurality of photodetectors provided to the optical fiber in the longitudinal direction. Detection light from a detection light emitter is input to the optical fiber, and allowed to propagate in the optical fiber, during which the individual photodetectors detect changes in the amount of light, with which the endoscope shape is estimated.

What is claimed is:

1. An endoscope insertion assisting system comprising:
one or more processors having hardware,
the processor being structured:
to acquire shape information of an endoscope insertion part during endoscopic examination; and
to execute, according to a priority condition, at least either one of
entering the shape information to an insertion status learning model and acquiring, from the insertion status learning model, an insertion status category resulted from categorization of insertion status of the endoscope insertion part, and
applying the shape information to a shape category determination logic and acquiring an insertion shape category resulted from categorization of insertion shape of the endoscope insertion part.

2. The endoscope insertion assisting system according to claim 1, wherein
the processor is structured:
to enter the shape information to the insertion status learning model and acquiring, from the insertion status learning model, an insertion status category resulted from categorization of insertion status of the endoscope insertion part;
to apply the shape information to the shape category determination logic and acquiring an insertion shape category resulted from categorization of insertion shape of the endoscope insertion part; and
in a case where the insertion shape category contained in the insertion status category does not match the insertion shape category acquired from the shape category determination logic, to correct the insertion shape category contained in the insertion status category, to the insertion shape category acquired from the shape category determination logic.

3. The endoscope insertion assisting system according to claim 1, wherein
the processor is structured to generate insertion assisting information with reference to the insertion status category, and to display the insertion assisting information on a monitor.

4. The endoscope insertion assisting system according to claim 1, wherein
the processor is structured to generate insertion control information with reference to the insertion status category.

5. The endoscope insertion assisting system according to claim 1, wherein
the processor is structured:
to acquire an endoscopic image during the endoscopic examination; and
to enter the endoscopic image to a site learning model and acquiring, from the site learning model, a site of a subject that appears in the endoscopic image.

6. The endoscope insertion assisting system according to claim 5, wherein
the processor is structured:
to enter the shape information to the insertion status learning model and acquiring, from the insertion status learning model, an insertion status category resulted from categorization of insertion status of the endoscope insertion part;
to enter an endoscopic image during the endoscopic examination to the site learning model and acquiring, from the site learning model, a site of a subject that appears in the endoscopic image; and
in a case where the site of the subject contained in the insertion status category does not match the site of the subject acquired from the site learning model, to correct the site of the subject contained in the insertion status category, to the site of the subject acquired from the site learning model.

7. The endoscope insertion assisting system according to claim 2, wherein
the processor is structured:
to acquire an endoscopic image during the endoscopic examination; and
to enter the shape information and the endoscopic image to the insertion status learning model and acquiring, from the insertion status learning model, the insertion status category.

8. The endoscope insertion assisting system according to claim 5, wherein
the processor is structured
to enter the endoscopic image and the shape information to the site learning model and acquiring, from the site learning model, the site of the subject that appears in the endoscopic image.

9. The endoscope insertion assisting system according to claim 1, wherein
the processor is structured:
to enter the shape information to the insertion status learning model and acquiring, from the insertion status learning model, an insertion status category resulted from categorization of insertion status of the endoscope insertion part;
to apply the shape information to the shape category determination logic and acquiring an insertion shape category resulted from categorization of insertion shape of the endoscope insertion part; and
to determine, with reference to the shape information, whether or not to correct the insertion shape category contained in the insertion status category, to the insertion shape category acquired from the shape category determination logic.

10. The endoscope insertion assisting system according to claim 1, wherein
the processor is structured:
to enter the shape information to the insertion status learning model and acquiring, from the insertion status learning model, an insertion status category resulted from categorization of insertion status of the endoscope insertion part;
to apply the shape information to the shape category determination logic and acquiring an insertion shape category resulted from categorization of insertion shape of the endoscope insertion part; and
to determine, with reference to priority information attached to at least either one of the insertion status category and the insertion shape category, whether or not to correct the insertion shape category contained in the insertion status category, to the insertion shape category acquired from the shape category determination logic.

11. The endoscope insertion assisting system according to claim 1, wherein
the processor is structured:
to enter the shape information to the insertion status learning model and acquiring, from the insertion status learning model, an insertion status category resulted from categorization of insertion status of the endoscope insertion part;
to apply the shape information to the shape category determination logic and acquiring an insertion shape category resulted from categorization of insertion shape of the endoscope insertion part; and
to determine, with reference to reliability of the insertion status category acquired from the insertion status learning model, whether or not to correct the insertion shape category contained in the insertion status category, to the insertion shape category acquired from the shape category determination logic.

12. The endoscope insertion assisting system according to claim 11, wherein
the processor is structured
in a case where the shape information satisfies none of the conditions of the shape category determination logic, and reliability of the insertion status category is lower than a threshold value, to determine that the insertion shape of the endoscope insertion part cannot be estimated.

13. The endoscope insertion assisting system according to claim 1, wherein
the shape category determination logic is a determination logic determined by a designer.

14. The endoscope insertion assisting system according to claim 6, wherein
the processor is structured:
to acquire temporally consecutive endoscopic images; and
in a case where the site of the subject to be detected by the site learning model is not detected consecutively for a predetermined time period, to invalidate the detected site of the subject.

15. The endoscope insertion assisting system according to claim 1, wherein
the processor is structured:
to acquire posture information of a subject; and
to correct the shape information with reference to the posture information.

16. The endoscope insertion assisting system according to claim 1, wherein
the processor is structured:
to acquire an endoscopic image during the endoscopic examination;
to determine a status regarding a distance between an endoscope distal end and an intestinal wall of a subject; and
to generate insertion assisting information or insertion control information, with reference to the insertion status category, and the status regarding the distance between the endoscope distal end and the intestinal wall of the subject.

17. The endoscope insertion assisting system according to claim 1, wherein
the processor is structured:
to acquire an endoscopic image during the endoscopic examination;
to determine a status regarding a distance between an endoscope distal end and an intestinal wall of a subject; and
to enter the shape information, the endoscopic image, and the status regarding the distance between the endoscope distal end and the intestinal wall of the subject, to the insertion status learning model and acquiring, from the insertion status learning model, the insertion status category.

18. The endoscope insertion assisting system according to claim 1, wherein
the processor is structured:
to acquire an endoscopic image during the endoscopic examination;
to determine a status regarding a residue or water; and
to generate insertion assisting information or insertion control information, with reference to the insertion status category, and the status regarding the residue or the water.

19. The endoscope insertion assisting system according to claim 1, wherein
the processor is structured to generate pain information that indicates pain occurred in a subject, with reference to the insertion shape of the endoscope insertion part and a site where an endoscope distal end resides, and to display the pain information on a monitor.

20. A method for controlling an endoscope insertion assisting system causing one or more processors having hardware:
to acquire shape information of an endoscope insertion part during endoscopic examination; and
to execute, according to a priority condition, at least either one of
entering the shape information to an insertion status learning model and acquiring, from the insertion status learning model, an insertion status category resulted from categorization of insertion status of the endoscope insertion part, and applying the shape information to a shape category determination logic and acquiring an insertion shape category resulted from categorization of insertion shape of the endoscope insertion part.

21. A storage medium that stores a program product causing a computer to execute:
acquiring shape information of an endoscope insertion part during endoscopic examination; and
executing, according to a priority condition, at least either one of
entering the shape information to an insertion status learning model and acquiring, from the insertion status learning model, an insertion status category resulted from categorization of insertion status of the endoscope insertion part, and
applying the shape information to a shape category determination logic and acquiring an insertion shape category resulted from categorization of insertion shape of the endoscope insertion part.
